(19) United States
(12) Patent Application Publication
(10) Pub. No.: US 2025/0163141 A1
(43) Pub. Date: May 22, 2025

(54) CCL4 ANTAGONIST FOR INHIBITING VASCULAR AGING

(71) Applicants: NATIONAL YANG MING CHIAO TUNG UNIVERSITY, Taipei City (TW); TAIPEI VETERANS GENERAL HOSPITAL, Taipei City (TW)

(72) Inventors: Ting-Ting CHANG, Taipei City (TW); Jaw-Wen CHEN, Taipei City (TW)

(73) Assignees: NATIONAL YANG MING CHIAO TUNG UNIVERSITY, Taipei City (TW); TAIPEI VETERANS GENERAL HOSPITAL, Taipei City (TW)

(21) Appl. No.: 18/953,859

(22) Filed: Nov. 20, 2024

(30) Foreign Application Priority Data
Nov. 22, 2023 (TW) 112145181
Aug. 20, 2024 (TW) 113131309

(51) Int. Cl. C07K 16/24; A61P 9/14

(52) U.S. Cl. CPC C07K 16/24; A61P 9/14; C07K 2317/76

(57) ABSTRACT

Provided is a method for inhibiting vascular aging in a subject in need thereof by administering a pharmaceutical composition including a CCL4 antagonist. The CCL4 antagonist inhibits vascular aging by reducing a CCL4 level, inhibiting a binding activity of CCL4 with a receptor thereof, or a combination thereof.

### SEQUENCE LISTING

The present application is being filed along with a Sequence Listing in electronic format. The Sequence Listing is provided as a file entitled 211445US-Sequence Listing.XML, created on May 3, 2024, which is 2,819 bytes (about 2.75 KB) in size. The information in the electronic format of Sequence Listing is incorporated herein by reference in its entirety.

### BACKGROUND

### 1. Technical Field

The present disclosure relates to a use of a chemokine C-C motif ligand 4 (CCL4) antagonist, especially to the use of the CCL4 antagonist in manufacturing a pharmaceutical composition for inhibiting vascular aging.

### 2. Description of Associated Art

As the extension of human lifespan, the life expectancy has increased from 33 years in 1900 to 70 years in current, showing the continuous aging of human population worldwide. Various organs undergo changes when a person experiences aging, with the changes in the cardiovascular system having the most considerable effects on health. As the age increases, physiologic changes in the cardiovascular system, including increases in the systolic and pulse pressures, thickening of the ventricle, and reduces in the cardiac diastolic function, the maximal heart rate, the maximal cardiac output, and the maximal oxygen consumption, will occur. Moreover, the decrease in vascular elasticity leads to dysfunction of the sinoatrial and atrioventricular nodes, which control the rhythm of cardiac contractions, due to a reduction in cell counts, resulting in an increase in the level of coagulation response factors.

Accordingly, once vascular aging occurs, it may lead to symptoms of systemic aging, including decreased physical and cardiovascular functions, and even fatal diseases such as myocardial infarction and stroke. However, there is no effective strategy to inhibit vascular aging in clinical practice currently.

Macrophage inflammatory protein-1β (MIP-1β, also referred to as CCL4 as used herein) is a member of the CC cytokine family, which is firstly isolated from a macrophage culture activated by lipopolysaccharides (Lodi P. J. et. al., Science 263: 1762-1767, 1994), and has the molecular weight of 7.8 kD and a protein structure consisting of a precursor with 92 amino acids. It has been observed in patients with a cardiovascular disease that the MIP-1β is upregulated (Tatara, Y. et. al, J Mol Cell Cardiol 47: 104-111, 2009; Mirabelli-Badenier, M. et. al., Thromb Haemost 105: 409-420, 2011).

MIP-1β/CCL4 performs biochemical functions thereof by binding to a CC cytokine (CCR, belonging to the G-protein-coupled receptor superfamily) on the surface of a cell. In an animal model of myocardial infarction, the generation of cytokine by inducing the infarcted heart promotes the replenishment of white blood cell populations with special properties to the ischemic site. In the myocardium of infarcted mice, MIP-1β and its receptor CCR5 will be significantly induced to produce (Dobaczewski M. et. al., Am J Pathol 176: 2177-2187, 2010). But up to date, no related study on whether the MIP-1β/CCL4 is related to vascular aging or not is provided.

Therefore, there is a need to clarify in clinical physical examination studies whether CCL4 is the key factor causing vascular aging and confirm whether inhibiting CCL4 can inhibit vascular aging, in order to develop new treatment strategies related thereto.

### SUMMARY

In view of the foregoing, the present disclosure provides a use of a CCL4 antagonist in manufacturing a pharmaceutical composition for inhibiting vascular aging, and the pharmaceutical composition comprises an effective amount of the CCL4 antagonist and a pharmaceutically acceptable carrier thereof.

In another aspect, the present disclosure provides a method for inhibiting vascular aging in a subject in need thereof, including administering an effective amount of a pharmaceutical composition to the subject, wherein the pharmaceutical composition includes a CCL4 antagonist and a pharmaceutically acceptable carrier thereof.

In an embodiment of the present disclosure, the CCL4 antagonist is at least one selected from the group consisting of an anti-CCL4 antibody or its fragment, a CCL4 RNA interference (RNAi) agent, a small molecular CCL4 antagonist, a CCR1 antagonist, a CCR2 antagonist, and a CCR5 antagonist.

In an embodiment of the present disclosure, vascular aging is accompanied with an increase in a CCL4 level.

In an embodiment of the present disclosure, the CCL4 antagonist inhibits vascular aging by reducing a CCL4 level, inhibiting a binding activity of the CCL4 with a receptor thereof, or a combination thereof.

In an embodiment of the present disclosure, the inhibition of vascular aging comprises resisting aging of vascular endothelial cells and/or enhancing an ability of angiogenesis.

In an embodiment of the present disclosure, the resisting aging of vascular endothelial cells comprises at least one selected from the group consisting of reducing ROS, reversing aging factors, and reducing inflammatory factors, and the enhancing the ability of angiogenesis comprises up-regulating angiogenic factors.

In one embodiment of the present disclosure, the reversing aging factor comprises at least one selected from the group consisting of down-regulating SIRT1, down-regulating p53, and down-regulating p16.

In an embodiment of the present disclosure, the inflammatory factor comprises at least one selected from the group consisting of TNF-α, IL-1β, and IL-6.

In an embodiment of the present disclosure, the ability of angiogenesis comprises at least one selected from the group consisting of vascularization, an ability of cell migration, and vascular germination.

In an embodiment of the present disclosure, the angiogenic factor comprises at least one selected from the group consisting of p-eNOS, p-AKT, VEGF, and SDF-1α.

In an embodiment of the present disclosure, the CCL4 antagonist is administrated at an amount of 0.01 mg/kg to 100 mg/kg.

In an embodiment of the present disclosure, the pharmaceutical composition is administrated 1 to 25 time(s) per week. In an embodiment of the present disclosure, the pharmaceutical composition is administrated continuously for 1 to 4 week(s).

In the present disclosure, via the inhibition function of the CCL4 antagonist, the aging factors, the inflammatory factors, and the angiogenic factors in vivo are regulated effectively, thereby resisting the aging of vascular endothelial cells and/or improving the ability of angiogenesis, achieving the efficacy of inhibiting vascular aging.

Other objectives, advantages and novel features of the invention will become more apparent from the following detailed description when taken in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

By reading the detail description below and referencing figures described herein, the implementation modes of the present disclosure will be further understood.

FIGS. 1A-1F show that inhibiting CCL4 can reverse the aging of cells by regulating aging factors and inflammatory factors. FIG. 1A shows the comparison of plasma CCL4 concentrations in young subjects (<30 years; n=7) and aged subjects (>55 years; n=8); FIG. 1B shows the effects of inhibiting CCL4 on aging-related galactosidase activity in young (P4) and aged (P9) cells (n=3); FIG. 1C shows effects of inhibiting CCL4 on the ROS in P4 and P9 cells (n=3); FIGS. 1D-1F show the effects of inhibiting CCL4 on expression of CCL4, aging factors and inflammatory factors in P4 and P9 cells, respectively (n=3). “N” represents cells cultured from “n” different individuals, and the cells cultured for each individual were subjected to three independent experiments. P4: Passage 4; P9: Passage 9; siCCL4: CCL4 siRNA; si-Control: non-target siRNA of 20-25 nt. *P<0.05; **P<0.01.

FIGS. 2A-2D show the effects of inhibiting CCL4 on the ability of angiogenesis. FIG. 2A shows that inhibiting CCL4 has no effect on cell proliferation (n=3); FIGS. 2B and 2C show the effects of inhibiting CCL4 on the vascularization and migration abilities of cells, respectively (n=3); and FIG. 2D shows the effects of inhibiting CCL4 on the expression of angiogenic factors in P4 and P9 cells (n=3).

FIGS. 3A-3E show the effects of inhibiting CCL4 with genetic knockout on the ability of angiogenesis in aged mice. FIG. 3A presents the representative images showing the effects of inhibiting CCL4 on aortic ring endothelial cells sprouting in young and aged mice; FIG. 3B shows the effects of inhibiting CCL4 on the area of vascular sprouts in young and aged mice (n=3); and FIGS. 3C-3E show the effects of inhibiting CCL4 on the expression of angiogenic factors, aging factors and inflammatory factors in young and aged mice (n=3). WT6M: wild-type mice at 6 months of age; CCL4KO6M: mice at 6 months of age with CCL4 gene-knockout; WT18M: wild-type mice at 18 months of age; and CCL4KO18M: mice at 18 months of age with CCL4 gene-knockout.

FIGS. 4A-4D illustrate that inhibiting CCL4 with genetic knockout enhances angiogenesis in aged mice. FIG. 4A presents the representative images showing the effects of inhibiting CCL4 on Matrigel plugs in young and aged mice and hemoglobin levels in the Matrigel plugs (n=6); FIG. 4B presents representative images of H&E-stained Matrigel plugs before and after inhibiting CCL4 in young and aged mice; and FIGS. 4C and 4D present representative images of Matrigel plugs immunostained with CD31 and Ki67 antibodies before and after inhibiting CCL4 in young and aged mice, respectively.

FIGS. 5A-5E illustrate the effects of inhibiting CCL4 with a neutralizing antibody on the ability of angiogenesis in aged mice. FIG. 5A presents the representative images showing the effects of inhibiting CCL4 on aortic ring endothelial cells sprouting in young and aged mice; FIG. 5B shows the effects of inhibiting CCL4 on the area of vascular sprouts in young and aged mice (n=6); and FIGS. 5C-5E show the effects of inhibiting CCL4 on the expression of angiogenic factors, aging factors and inflammatory factors in young and aged mice (n=3). WT6M: wild-type mice at 6 months of age; WT18M: wild-type mice at 18 months of age; and mAb: monoclonal neutralizing antibody.

FIGS. 6A-6D illustrate that inhibiting CCL4 with a neutralizing antibody enhances angiogenesis in aged mice. FIG. 6A presents the representative images showing the effects of inhibiting CCL4 on Matrigel plugs in young and aged mice and hemoglobin levels in the Matrigel plugs (n=6); FIG. 6B presents representative images of H&E-stained Matrigel plugs before and after inhibiting CCL4 in young and aged mice; and FIGS. 6C and 6D present representative images of Matrigel plugs immunostained with CD31 and Ki67 antibodies before and after inhibiting CCL4 in young and aged mice, respectively.

### DETAILED DESCRIPTION OF THE PREFERRED EMBODIMENTS

The following examples are used to illustrate the present disclosure. One having ordinary skill in the art can easily conceive of the other advantages and effects of the present disclosure based on the invention of the specification. The present disclosure can also be implemented or applied as described in various examples. It is possible to modify or alter the following examples for carrying out the present disclosure without violating its spirit and scope, for different aspects and applications.

Note, as used herein, the terms “a,” “an” and “the” of singular form include a plurality of referents unless clearly limited to one referent. The terms “or” and “and/or” can be used interchangeably, unless it is otherwise indicated explicitly in the context.

As used herein, the term “comprise” or “include” refers to the composition, method and corresponding components which are essential to the present disclosure, but is open for encompassing unspecified elements regardless whether being essential.

The terms “individual” and “subject” are used interchangeably in the present disclosure, and the term “subject” refers to a human or an animal. Examples of the subject includes, but not limited to, human, monkey, mouse, rat, marmot, ferret, rabbit, hamster, cattle, horse, pig, deer, dog, cat, fox, wolf, chicken, emu, ostrich and fish. In certain embodiments of the present disclosure, the subject is a mammal, e.g., a primate, such as a human.

The present disclosure relates to a use of CCL4 antagonist in manufacturing pharmaceutical composition for inhibiting vascular aging. In an embodiment of the present disclosure, the pharmaceutical composition comprises an effective amount of CCL4 antagonist and a pharmaceutically acceptable carrier thereof.

In an embodiment of the present disclosure, the CCL4 antagonist can prevent CCL4 from binding to the receptor thereof. In another embodiment, the CCL4 antagonist is a reagent for inhibiting intracellular signaling generated by the binding of CCL4 with the receptor thereof. For example, the CCL4 antagonist can target at least one of CCL4 and the receptor of CCL4, thereby blocking the signaling related to CCL4. As used herein, the receptor of CCL4 includes, but not limited to, CCR1, CCR2 and CCR5.

As used herein, the terms “CCR1” or “CCR1 receptor,” “CCR2” or “CCR2 receptor,” and “CCR5” or “CCR5 receptor” can be used interchangeably and have the general meanings thereof in the art. CCR1, CCR2, and CCR5 receptors can be derived from any source, but generally are CCR1, CCR2, and CCR5 receptors derived from mammals (e.g., human or non-human primates). In some embodiments of the present disclosure, the CCR1, CCR2, and CCR5 receptors are human receptors.

In an embodiment of the present disclosure, the term “CCL4 antagonist” includes any entity which, after being administrated to a subject, results in inhibition or down-regulation of CCL4-related biological activities in the subject, including any downstream biological effect other than one caused by the binding of CCL4 and the receptor thereof. The CCL4 antagonist includes any reagent capable of inhibiting CCL4 activity or blocking activation of a CCL4 receptor or any biological effect downstream to the activation of a CCL4 receptor. For example, the reagent can be a small organic molecule or an antibody against CCL4, such as a CCL4 neutralizing antibody, which can block the interaction of CCL4 with a receptor thereof or can block the activity of CCL4. The CCL4 antagonist can also be a small molecule or an antibody against a CCL4 receptor to prevent the receptor from accessing its ligand CCL4.

In an embodiment of the present disclosure, the CCL4 antagonist is at least one selected from the group consisting of an anti-CCL4 antibody or its fragment, a CCL4 RNA interference agent, a small molecular CCL4 antagonist, a CCR1 antagonist, a CCR2 antagonist, and a CCR5 antagonist.

One having the ordinary skill in the art can convert and obtain the effective dose for human or other animals from a dosing test of animals (such as mice) according to pharmacokinetics. In an embodiment of the present disclosure, the effective amount of the CCL4 antagonist per kg body weight for administration to the subject has a lower limit selected from 0.01 mg/kg, 0.05 mg/kg, 0.1 mg/kg, 0.5 mg/kg, 1 mg/kg, 2 mg/kg, 2.5 mg/kg, 5 mg/kg, 10 mg/kg, 15 mg/kg, 20 mg/kg, and 25 mg/kg, and an upper limit selected from 1000 mg/kg, 900 mg/kg, 800 mg/kg, 700 mg/kg, 600 mg/kg, 500 mg/kg, 400 mg/kg, 300 mg/kg, 200 mg/kg, 100 mg/kg, 90 mg/kg, 80 mg/kg, 70 mg/kg, 60 mg/kg, 50 mg/kg, 40 mg/kg, and 30 mg/kg. In some embodiments, the CCL4 antagonist is administrated preferably at an amount of 0.01 mg/kg to 100 mg/kg; more preferably, the CCL4 antagonist is administrated at an amount of 2 mg/kg to 10 mg/kg; and still more preferably, the CCL4 antagonist is administered at an amount of 2.5 mg/kg to 5 mg/kg.

It is a technical proposal known by one having the ordinary skill of the art to adjust the administration frequency according to the components or dose concentrations of the pharmaceutical composition and the subject being administrated to. In an embodiment of the present disclosure, the pharmaceutical composition containing the CCL4 antagonist is administered 1 to 4 time(s) per day. In another embodiment, the pharmaceutical composition containing the CCL4 antagonist is administered 1 to 25 time(s) per week. For example, the pharmaceutical composition containing the CCL4 antagonist is administrated twice per day or 5 times per week. In an embodiment of the present disclosure, the pharmaceutical composition containing the CCL4 antagonist is administrated 1 to 5 time(s) per week, or 2 to 7 time(s) per week.

In an embodiment of the present disclosure, the pharmaceutical composition containing the CCL4 antagonist is administrated continuously for 1 to 4 week(s) to inhibit vascular aging. In another embodiment of the present disclosure, the pharmaceutical composition containing the CCL4 antagonist is administrated continuously for 1 to 8 week(s) to inhibit vascular aging.

In an embodiment of the present disclosure, the primary factor causing aging is the aging of vascular endothelial cells, which leads to the imbalance between vasodilators and vasoconstrictors. Moreover, during aging, the imbalance and dysfunction of the antioxidant system occurs due to the disability of cells for continuous division and regeneration, resulting in an increase in the level of reactive oxygen species (ROS) in cells. The increase in ROS results in excessive CCL4 secreted by endothelial cells and reduced secretion of vascular endothelial growth factor (VEGF) and stromal derived factor-1α (SDF-1α) in endothelial cells, which in turn result the dysfunction of endothelial cells.

In an embodiment of the present disclosure, the vascular aging is accompanied with an increase in CCL4 level.

In an embodiment of the present disclosure, the CCL4 antagonist inhibits the vascular aging by reducing CCL4 level, inhibiting the binding activity of CCL4 with receptor thereof, or combination thereof.

In an embodiment of the present disclosure, the inhibiting vascular aging comprises resisting aging of vascular endothelial cells and/or enhancing the ability of angiogenesis.

As used herein, the term “vascular endothelial cells” consists of a layer of flat cells forming the inner wall of a blood vessel, which is the interface between the intraluminal blood in the blood vessel and other blood vessel wall tissues. Endothelial cells distribute along the entire circulating system from heart to the smallest microvasculature. The normal function of endothelial cells is crucial for maintaining systemic vascular tension, inhibiting platelet aggregation, regulating immune responses, and inhibiting vascular smooth muscle proliferation. The abnormal function of endothelial cells is also the early indicator for occurrence of various cardiovascular diseases.

In an embodiment of the present disclosure, the resisting aging of vascular endothelial cells comprises at least one selected from the group consisting of reducing ROS, reversing aging factors, and reducing inflammatory factors, and the enhancing the ability of angiogenesis comprises up-regulating angiogenic factors.

As used herein, the term “ROS (Reactive oxygen species)” is byproducts during an aerobic metabolism process of an organism, and includes oxygen ions, peroxides, and oxygen-containing free radicals. The particles are considerable small and very active due to the presence of unpaired free electrons. Excessively high levels of reactive oxygen species can cause damage to cell and gene structures. ROS are natural byproducts from oxygen after a normal oxygen metabolism process, and function greatly in cell signaling and in maintaining homeostasis. However, the levels of ROS increase dramatically under the influence of time and external environment. The reason causing such a change can be significant damage to cell structures.

As used herein, the term “aging factor” refers to an aging indicator on mammal cells, the level of which is in positive or negative correlation to aged cells. In other words, in some embodiments, the level of aging factor up-regulates as cell aging; and in some further embodiments, the level of aging factor down-regulates as cell aging. On the contrary, the level of aging factors described above will be regulated reversely when the aging phenomena are reversed.

In an embodiment of the present disclosure, the reversing aging factors comprises at least one selected from up-regulating SIRT1 (Sirtuin 1, also referred as NAD-dependent deacetylase Sirtuin-1), down-regulating p53, and down-regulating p16.

As used herein, the term “inflammatory factors” refers to various cytokines involving in an inflammatory response, such as pro-inflammatory cytokines. A general inflammatory response can provide self-defense and repair for body, but chronic inflammation or inflammation imbalance can cause various diseases, produce pro-inflammatory cytokines to activate immune cells, release a large number of damage-associated molecular pattern (DAMPs), and affect the patient's whole body through blood circulation, triggering an inflammatory response.

In an embodiment of the present disclosure, the inflammatory factor comprises at least one selected from the group consisting of TNF-α (tumor necrosis factor-α), IL-1β (interleukin-1β), and IL-6 (interleukin-6).

In an embodiment of the present disclosure, the ability of angiogenesis comprises at least one selected from the group consisting of vascularization, cell migration ability, and vascular germination.

As used herein, the term “vascularization” represents the growth and formation of blood vessels. Angiogenesis comprises growing new blood vessels from existing ones, and refers to vascularization in spontaneous formation of blood vessels and neovascularization through splitting of existing ones. Angiogenesis comprises “neovascularization,” “revascularization,” “neoangiogenesis,” and “blood vessel regeneration.”

As used herein, the term “cell migration” refers to the movement of a cell upon receiving a signal for migration or sensing a concentration gradient of certain substances. During the movement, cells continuously repeat the process of extending synapses/pseudopodia forwards and then pulling the posterior cell body. The cytoskeleton and its binding proteins, as well as the intercellular substances are material basis for this process, and there are further various substances can regulate this process precisely. During angiogenesis, a population of vascular endothelial cells move outwards from the gathering site and differentiate. In other words, cell migration is essential to the process of angiogenesis.

As used herein, the term “vascular germination” is also referred to sprouting angiogenesis or sprouting-type angiogenesis, which is the firstly found mode of angiogenesis. Vascular germination can be divided into three stages well studied. First, a tissue lacking vasculature will be in a hypoxic status, and in turn releases signaling molecules such as vascular endothelial growth factor A (VEGF-A). Then, the secreted signaling molecules, such as VEGF-A, activate receptors on the existing vascular endothelial cells to activate the vascular endothelial cells. These activated vascular endothelial cells are also referred to as tip cells and can release protease used for degrading the basilar membrane to allow the endothelial cells to fall off from the existing (parent) vascular walls. Finally, the endothelial cells fallen off from the existing blood vessels proliferate in surrounding stroma to form solid sprouts connecting adjacent blood vessels. Cells behind the tip cells proliferate continuously to make the newly generated microvascular sprouts to grow continuously, and these cells are also called stalk cells. Stalk cells proliferate rapidly and contribute mainly to form branched networks of the newly generated blood vessels. The main determinant for vascular endothelial cells to become tip cells or stalk cells includes the relative amount between different signaling molecules including VEGF, JAG-1, etc. in the microenvironment where the cells are.

As used herein, the term “angiogenic factor” refers to a factor associated with the physiology promoting angiogenesis. In an embodiment, the angiogenic factor comprises one selected from the group consisting of p-eNOS (p-endothelial nitric oxide synthase), p-AKT (p-protein kinase B), VEGF (vascular endothelial growth factor), SDF-1α (stromal cell-derived factor-1α), and combination thereof.

As used herein, the term “effective amount” refers to an amount which is required for an activator (e.g., a CCL4 antagonist) to impart desired therapeutic effects (e.g., desired inhibition of vascular aging) for a subject being treated. As known by one having ordinary skill in the art, the effective amount will vary according to the route of administration, the use of excipients, the possibility of use in combination with other treatments, and the disorder to be treated.

In an embodiment of the present disclosure, “pharmaceutically acceptable carrier” can be a diluent, a disintegrant, a binder, a lubricant, a flow aid, a surfactant, or a combination thereof.

In one embodiment of the present disclosure, the term “pharmaceutical composition” can be a sterilized injectable composition, which can be a solution or a suspension in a non-toxic parenterally acceptable diluent or solvent. The acceptable diluent and solvent which can be employed comprise 1,3-butanediol, mannitol, water, Ringer's solution, and isotonic sodium chloride solution. Moreover, non-volatile oil is generally used as a solvent or a suspending medium (such as synthetic glycerin monoester or glycerin diester). Fatty acids, such as oleic acid and its derivatives of glycerin esters, can be used in formulating an injectable agent, as a pharmaceutically acceptable natural oil (e.g., olive oil or castor oil), for example, in its polyoxyethylated form. Such solutions or suspensions in oil can also comprise long chain alcohol diluents or dispersing agents, carboxymethylcellulose or similar dispersing agent. Other commonly used surfactants (e.g., Tweens and Spans and other similar emulsifying agents or bioavailability enhancers) are used for preparation of pharmaceutically acceptable solid, liquid or other dosage forms, for the purpose of formulation.

Carriers in a pharmaceutical composition must be “acceptable,” which is compatible with ingredients in the composition (and can stabilize the ingredients) and isn't harmful to a subject to be treated. One or more cosolvent(s) can be used as pharmaceutical excipient(s) to deliver an active compound. Other examples of carrier further comprise colloidal silica, magnesium stearate, cellulose, and sodium lauryl sulfate.

The present disclosure has been illustrated through various examples, and the examples below are only illustrative and are not intended to limit the scope of application of the present disclosure in any way.

### EXAMPLES

### Materials and Methods

### Clinical Samples

Blood samples were taken from peripheral veins of volunteer subjects, with the subjects having diabetes, major systemic diseases and major surgery in the past 6 months being excluded. Demographic and clinical data were obtained at the time of enrollment of the trial. The human research described is approved by the research committee and complies with the Declaration of Helsinki.

### Cell Culturing

Primary HAECs (ScienCell, Catalog #6100, Carlsbad, CA, USA) were cultured in a culturing plate coated with fibronectin in a culture medium containing 5% fetal bovine serum and 1% endothelial cells growth supplement at 37° C. in an environment of 95% air and 5% carbon dioxide.

siRNA Transfection

Cells were transfected with ccl4 siRNA (Santa Cruz Biotechnology, sc-43932, Dallas, TX, USA) in a culture medium by using Lipofectamine 2000 (Invitrogen, Carlsbad, CA, USA).

### Cell Proliferation Assay

Cell viability was evaluated by using Cell Counting Kit-8 (CCK-8; Dojindo Molecular Technologies, Inc., Rockville, MD, USA) according to instruction of the manufacturer. In brief, a reagent was added and cells were cultured at 37° C. for 2 hrs, and then the absorbance at 450 nm was determined.

### β-Galactosidase Staining Assay

The aging phenotypes were detected by using a β-galactosidase staining kit (Merck, Darmstadt, Germany). The number of β-galactosidase-positive cells associated with aging was observed in 10 low power fields randomly selected through an optical microscope.

### ROS Generation Assay

Hydrogen peroxide generated by cells was determined by using Amplex Red Hydrogen Peroxide/Peroxidase Assay Kit (Invitrogen, Carlsbad, CA, USA). In brief, a stoichiometric reaction (consisting of water and resorufine) of Amplex Red and hydrogen peroxide (H₂O₂) was catalyzed by horseradish peroxidase. Then, the cells were scraped into a phosphate buffer (pH 7.4). The cell debris was centrifuged and 50 L of work solution was added to 50 μL of supernatant. After incubating for 30 minutes, fluorescence at 540 nm exciting wavelength and 590 nm emitting wavelength were measured on a microplate analyzer.

### Migration and Vascularization Experiments

Migration was evaluated by a chamber assay. Cells (1×10⁴ cells) were resuspended in a culture medium containing 5% FBS. Cells were added to the upper chambers of a 24-well Transwell with a polycarbonate membrane. A culture medium supplemented with fetal bovine serum was added into the bottom chambers and each chamber was incubated for 18 hrs. Then, the membrane was fixed with 4% paraformaldehyde and stained with hematoxylin solution. The number of moved cells was counted in a random high power (×100) microscope field.

In vitro vascularization assay was performed by using an angiogenesis assay kit (Invitrogen, Carlsbad, CA, USA). ECMatrix gel solution was mixed with ECMatrix dilution buffer and placed in a 96-well plate. Thereafter, cells (1×10⁴ cells) were placed in a matrix solution containing 10% FBS medium and incubated for 16 hrs. Vascularization was inspected under an inverted optical microscope (×40). Image-Pro Plus (Media Cybernetics, Inc. Rockville, MD, USA) was used for comparing the average value of number of complete vessels formed by the cells.

### Western Blotting

The total cells or tissue lysate were extracted with a lysis buffer and isolated for proteins in 8-12% (v/v) SDS-PAGE gel. After electrophoresis (Bio-Rad Laboratories, Hercules, CA, USA), the proteins were transferred onto a nitrocellulose membrane (Millipore, Darmstadt, Germany). The membrane was incubated at 4° C. overnight with the antibodies against: CCL4 (Santa Cruz Biotechnology, sc-393441, Dallas, TX, USA), p-AKT (BD Biosciences, 550747; NJ, USA), AKT (BD Biosciences, 610868; NJ, USA), VEGF (Santa Cruz Biotechnology, sc-152; Dallas, TX, USA), SDF-1 (Cell Signaling, 3530S; Boston, MA, USA), IL-1β (Santa Cruz Biotechnology, sc-7884; Dallas, TX, USA), IL-6 (Cell Signaling, 12153S; Boston, MA, USA), TNF-α (Cell Signaling, 3707S; Boston, MA, USA), SIRT1 (Cell Signaling, 8469S; Boston, MA, USA), p53 (Cell Signaling, 2524S; Boston, MA, USA), p16 (Cell Signaling, 80772S and 2927IS; Boston, MA, USA), p-eNOS (Cell Signaling, 957IS; Boston, MA, USA), and eNOS (Cell Signaling, 32027S; Boston, MA, USA); as well as actin (Merck, MAB1501, Darmstadt, Germany). The proteins described above were normalized to expression level of actin.

### Preparation of Animals

6-Week aged male C57BL/6JNarl-Ccl4eml-knockout (CCL4KO) mice were purchased from National Experimental Animals Center (Taipei, Taiwan). CCL4KO mice were generated in C57BL/6JNarl genetic setting by using a CRISPR/Cas9 system. All mice were genotyped by PCR using specific primers (forward primer: 5′-TCTCCCTCCTTTCTCTTCCGTG-3′ (SEQ ID NO:1); reverse primer: 5′-TCTACTCCCAATGATGGCTGACC-3′ (SEQ ID NO:2)). C57BL/6JNarl mice were used as wild-type (WT) controls. Mice at 6 months of age were defined as youth, and mice at 18 months of age were defined as aged. According to the regulations of the Animal Protection Committee of National Yang Ming Chiao Tung University, animals were kept under specific pathogen free conditions at the Animal Center of National Yang Ming Chiao Tung University (Taipei, Taiwan), and all mice were kept in miniature isolation cages with a 12-hour day night cycle. The animal research has been approved by the Animal Protection Committee of National Yang Ming Chiao Tong University.

Treatment with a Neutralizing Antibody

C57BL/6JNarl wild-type (WT) mice of 6 and 18 months old were intraperitoneally injected with a CCL4 monoclonal neutralizing antibody, respectively (R&D Systems, MAB451, Minneapolis, MN, USA). Injection dose: 100 μg/time; injection frequency: 3 times per week for 2 weeks.

### Matrigel Angiogenesis Experiments

Mice were subcutaneously injected with growth factor-reduced Matrigel (Corning® Matrigel, Glendale, AZ, USA) containing 30 ng/mL VEGF (Peprotech, Rocky Hill, CT, USA) and 50 U heparin (Sigma-Aldrich, Darmstadt, Germany). After 14 days, the Matrigel plugs were collected, homogenized in 500 L of cell lysate, and centrifuged at 4° C. and 6000 g for 60 minutes. Hemoglobin was detected at the wavelength of 400 nm by using a colorimetric assay (Sigma-Aldrich, MAK115, Darmstadt, Germany). Moreover, the Matrigel plugs obtained were used for histological and immunohistological analysis.

### Aortic Ring Assay

Aortic rings were cut into 0.5 mm sections and embedded in 1 mg/mL of type 1 rat tail collagen matrix (Millipore, Darmstadt, Germany), and were incubated at 37° C. for 1 hr. Aortic rings were cultured in a 24-well plate in EBM-2 (Lonza, Basel, Switzerland) containing 2.5% fetal bovine serum (Gibco, Carlsbad, CA, USA), 50 U/mL penicillin and 0.5 mg/mL streptomycin (Sigma-Aldrich, Darmstadt, Germany) with 30 ng/mL VEGF (Peprotech, Rocky Hill, CT, USA) for 7 days. Images were taken with a microscope (×100).

### Capillary Densities and Cell Prolifereation in Matrigel Plugs

Hematoxylin/eosin (H&E) staining was employed to evaluate the changes in morphologies. Sections were deparaffinized and incubated with an anti-murine CD31 rat monoclonal antibody (Abcam, 124432, Waltham, MA, USA) and an anti-murine proliferative marker Ki67 rabbit polyclonal antibody (Novus, NB500-170, Minneapolis, MN, USA). The distribution of the antibodies was visualized by using an avidin-biotin complex technique and Vector Red developing matrix, and then was counterstained with hematoxylin.

### Statistical Analysis

Results were presented in the form of mean±SD. The statistical analysis was done by unpaired Student t test or ANOVA followed by Scheffe multiple comparison post-hoc test. SPSS software (version 14; SPSS) was used for data analysis. p Value <0.05 was considered statistically significant.

### Example 1: Plasma CCL4 Concentration in Aged Subjects Increased

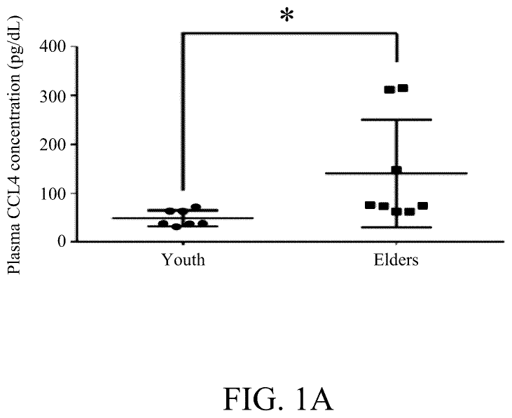

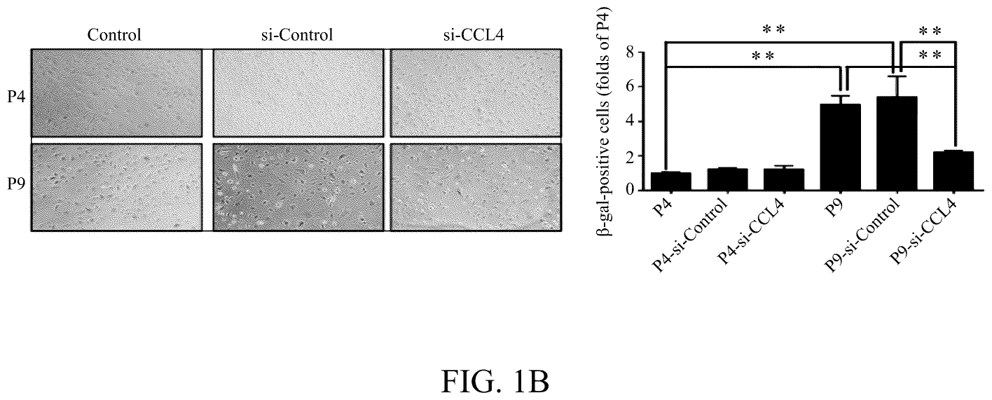

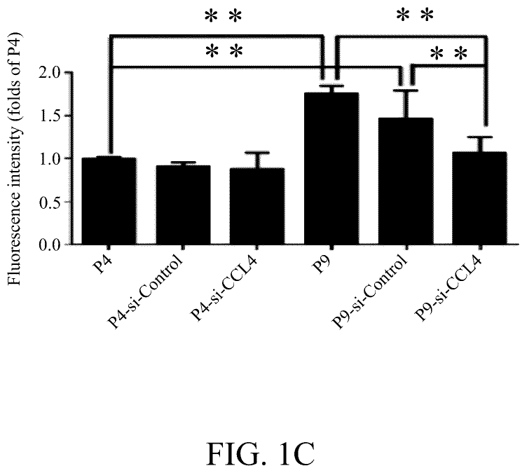

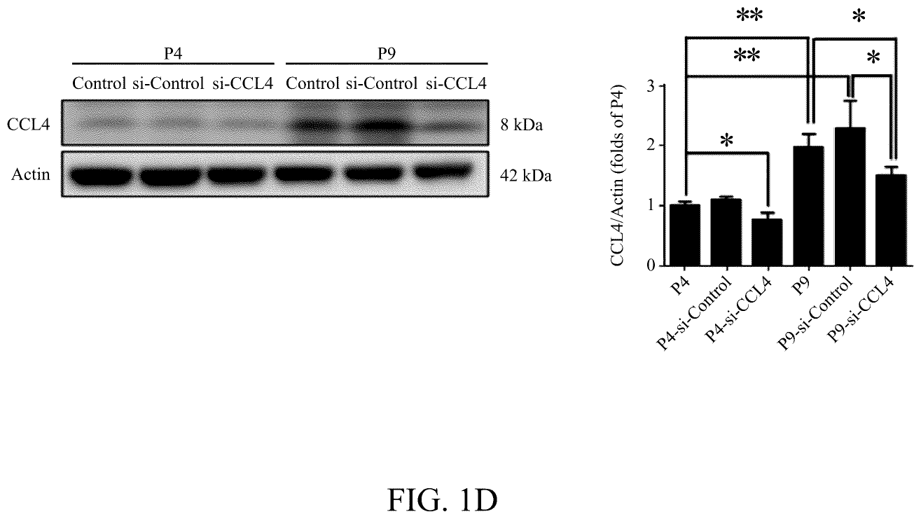

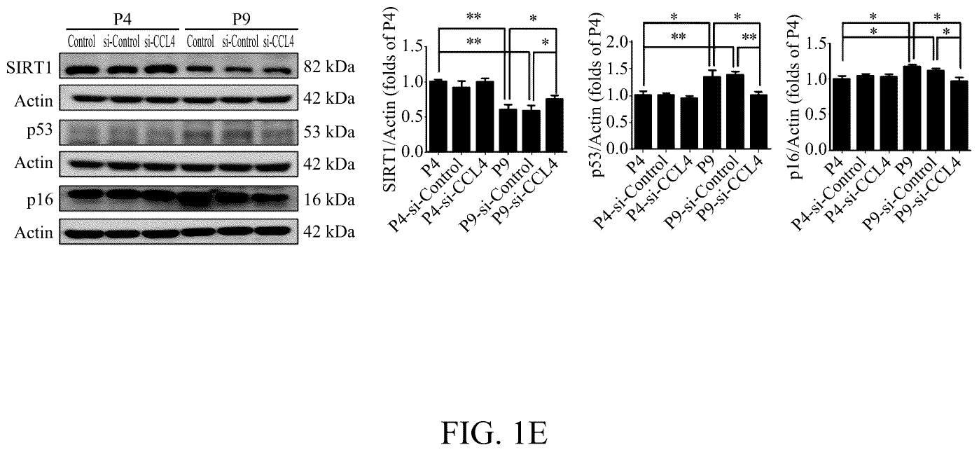

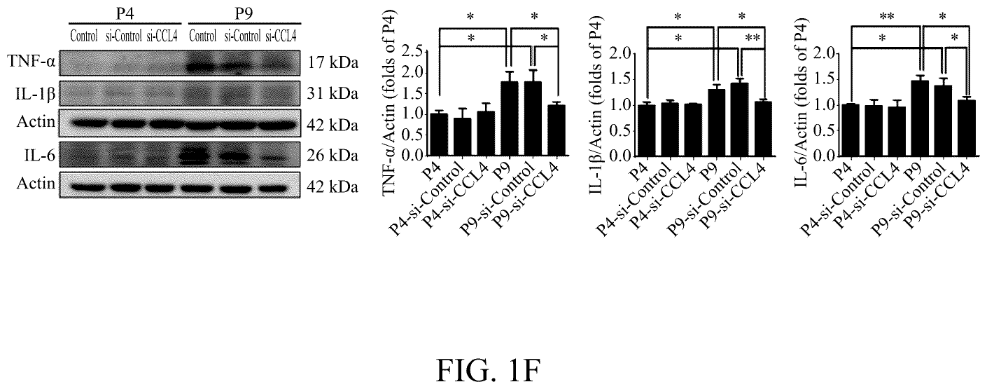

Plasmas were taken from young (<30 years) and aged (>55 years) subjects. Compared with the youth, the aged subjects had increases in plasma CCL4 levels (FIG. 1A), suggesting that CCL4 may be associated with systemic and vascular aging processes.

### Example 2: Inhibition of CCL4 can Reverse Cell Aging and Reduce Inflammation of Aged HAECs

For human aortic endothelial cells (HAECs), cell aging in aged cells (Passage 9; P9) increased significantly, as compared with in young cells (Passage 4, P4). It had been confirmed that inhibiting CCL4 with siRNA decreased aging of HAECs (FIG. 1B); generation of ROS in aged HAECs increased, and generation of ROS decreased in the group treated with siCCL4 (FIG. 1C); the expression level of CCL4 was higher in aged HAECs and was reduced by administration of siCCL4 (FIG. 1D); and inhibiting CCL4 reversed aging markers, e.g., up-regulated expression level of SIRT1 and down-regulated that of p53 and p16 (FIG. 1E). In addition, inhibition of CCL4 decreased age-induced inflammatory proteins, such as TNF-α, IL-1β and IL-6 (FIG. 1F). These data showed that inhibiting CCL4 reversed the aging process and decreased inflammation of aged HAECs.

### Example 3: Inhibition of CCL4 Reversed Age-Induced Cell Dysfunction and Increased Angiogenic Factors in Aged HAECs

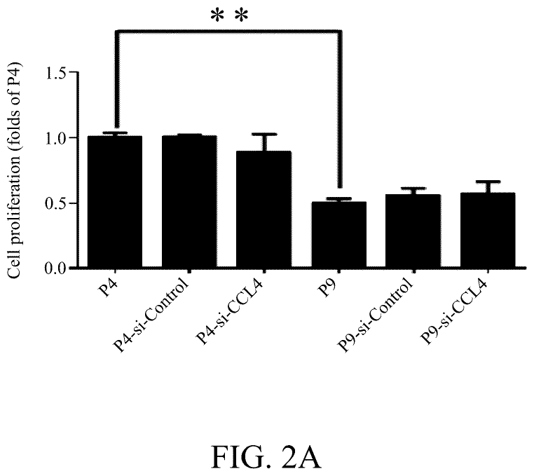

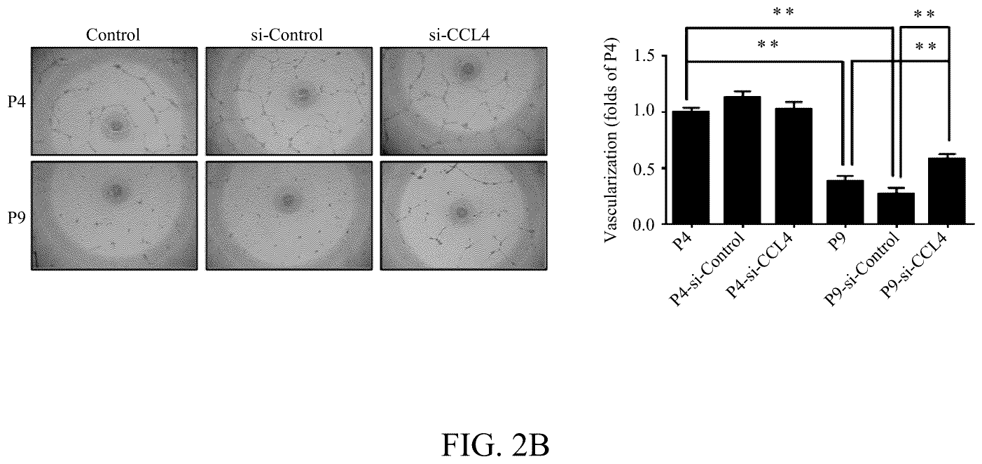

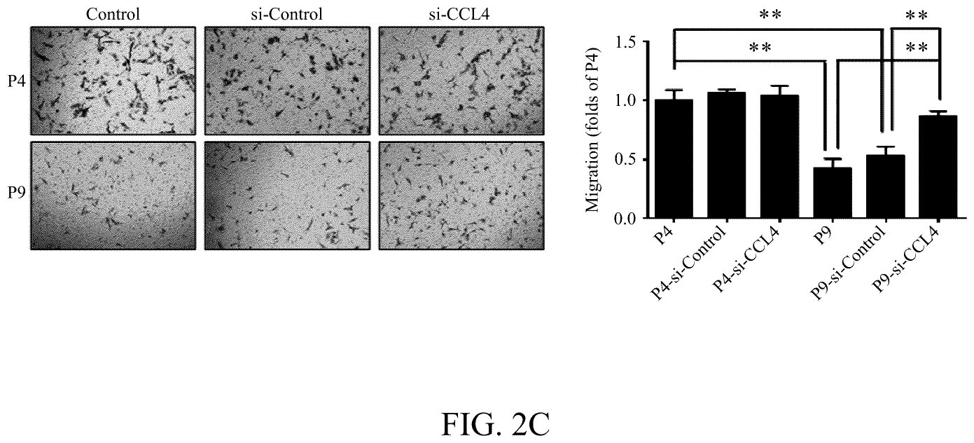

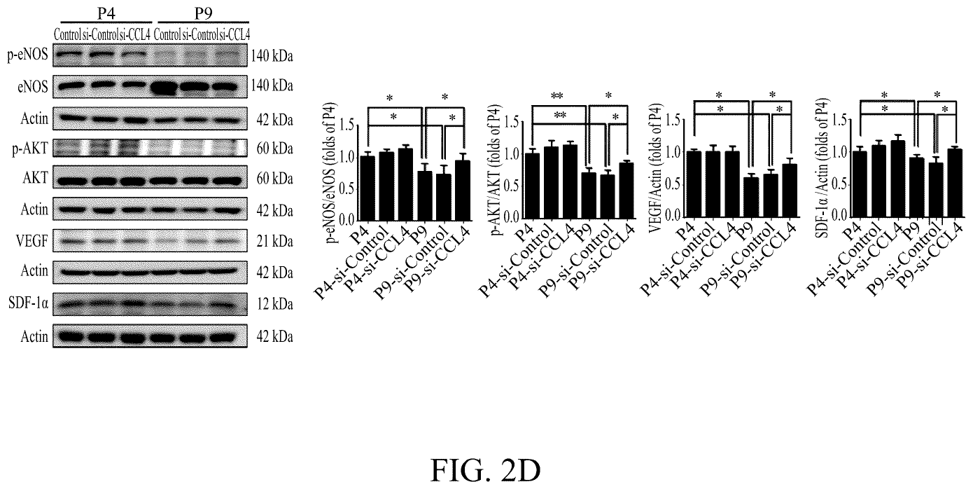

The ability of cell proliferation in aged HAECs reduced. It had been confirmed that inhibiting CCL4 with siRNA didn't affect cell proliferation in aged HAECs (FIG. 2A); the abilities of vascularization and migration of aged HAECs decreased and were significantly improved by inhibition with siCCL4 (FIGS. 2B and 2C); the angiogenic factors including p-eNOS, p-AKT, VEGF and SDF-1α, etc. in aged HAECs decreased and were up-regulated in the groups of inhibiting CCL4 (FIG. 2D). The results described above showed that inhibiting CCL4 reversed functions of aging-damaged cells and increased angiogenic factors in aged HAECs.

### Example 4: Angiogenesis in Aged Mice can be Improved by Inhibiting CCL4 Through Gene Knockout

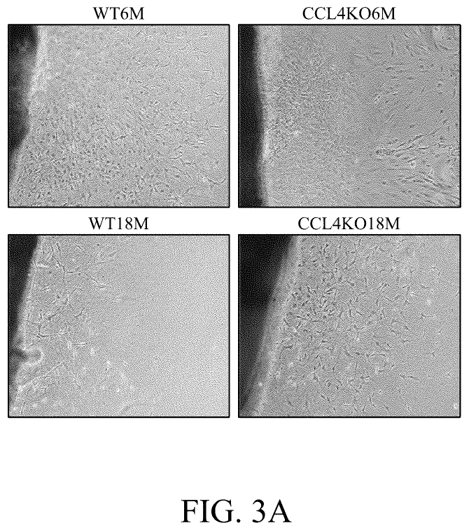

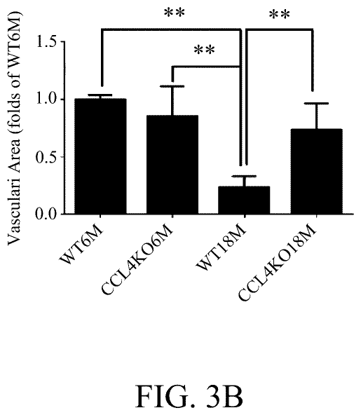

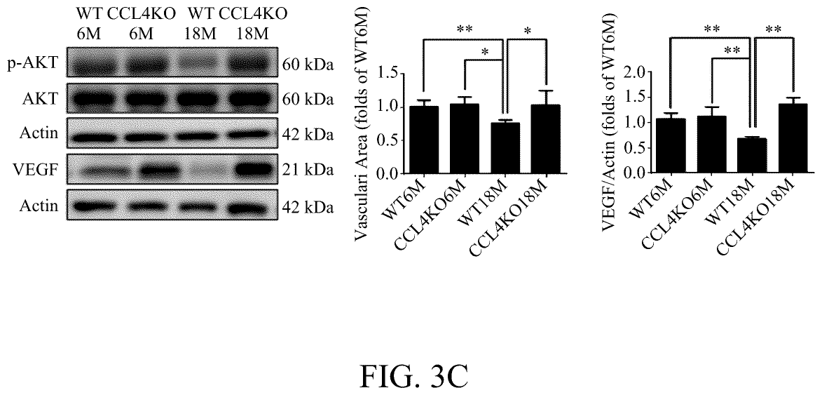

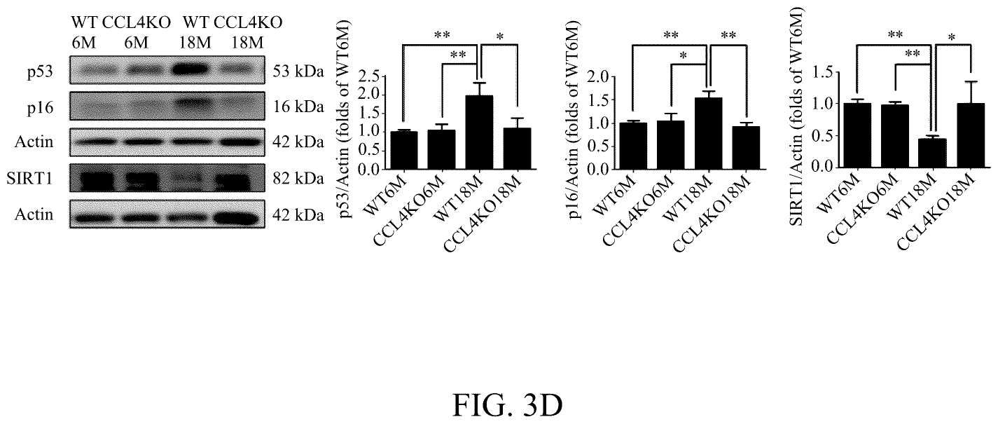

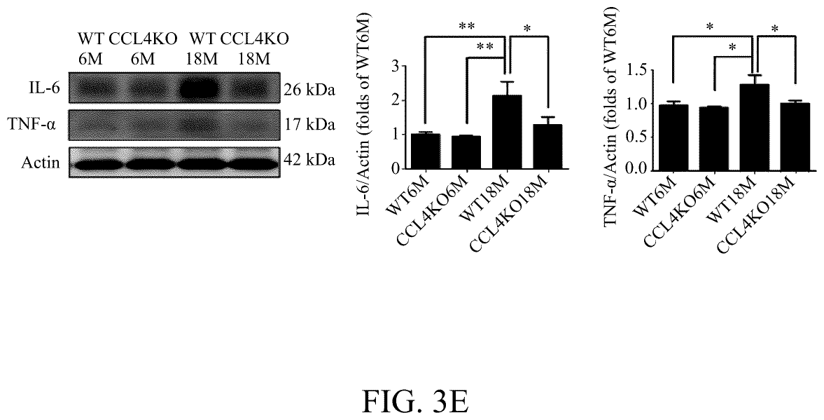

Aorta sprouting from aortic rings was damaged in aged wild-type mice (WT18M), as compared with young mice (WT6M). The area of aortic ring vascular sprouting in aged CCL4-knockout mice (CCL4KO18M) increased, as compared with aged wild-type mice (WT18M) (FIGS. 3A and 3B). In addition, in the evaluation of the expression of angiogenic, aging, and inflammatory markers in aortic tissues, the aortas of aged CCL4-knockout mice (CCL4KO18M) has a higher level of angiogenic factors (p-AKT/VEGF) (FIG. 3C), as well as reversed aging proteins (up-regulated SIRT1/down-regulated p53/down-regulated p16) (FIG. 3D) and a lower level of inflammatory proteins (TNF-α/IL-6) (FIG. 3E), as compared with aged wild-type mice (WT18M).

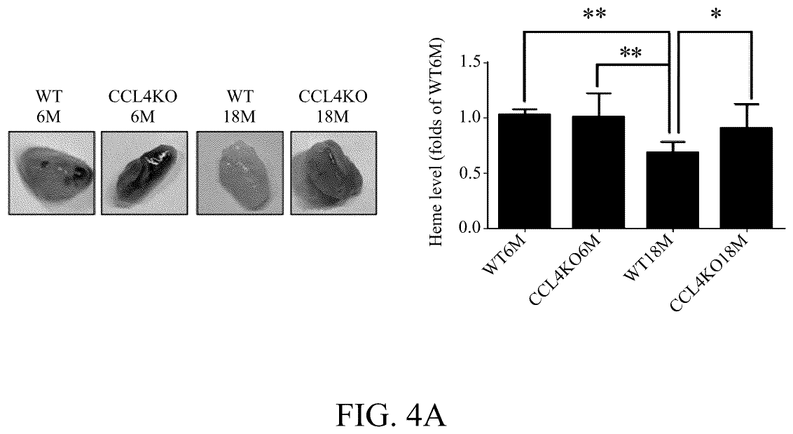

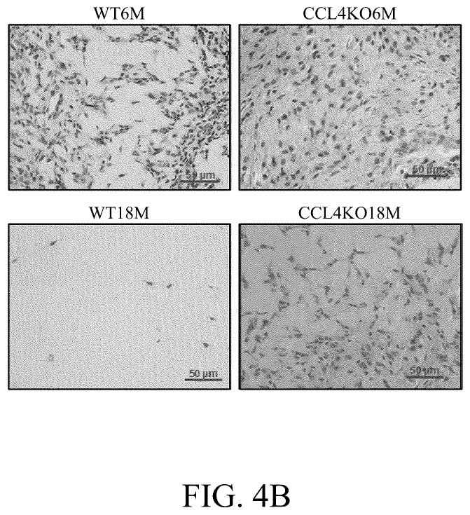

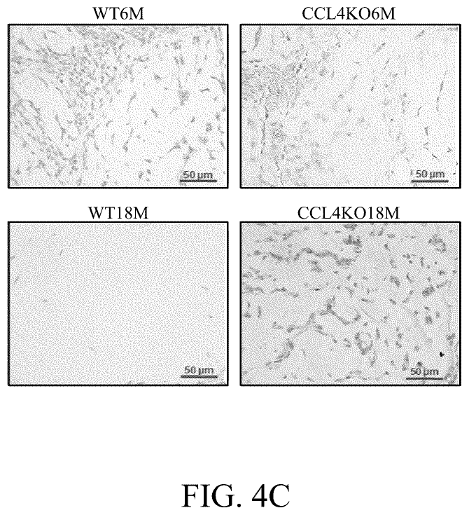

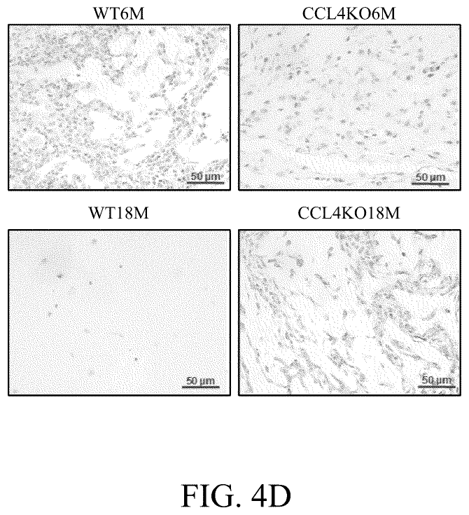

Moreover, the effect of CCL4 knockout on neovascularization was confirmed by Matrigel plug assay in vivo. Lower levels of vascularization and hemoglobin were observed in Matrigel plugs of aged wild-type mice (WT18M), as compared with those in Matrigel plugs of young mice (WT6M). The levels of vascularization and hemoglobin in Matrigel plugs of aged CCL4-knockout mice (CCL4KO18M) increased, as compared with aged wild-type mice (WT18M) (FIG. 4A). Increases in the numbers of blood vessels in Matrigel plugs of aged CCL4-knockout mice (CCL4KO18M) were also observed in hematoxylin-eosin (H&E) and CD31 staining assay (FIGS. 4B and 4C). In addition, in Matrigel plugs of aged CCL4-knockout mice (CCL4KO18M), expression of Ki67 (a marker for cell proliferation) at a level higher than that in aged wild-type mice (WT18M) was observed (FIG. 4D).

### Example 5: Angiogenesis in Aged Mice can be Improved by Inhibiting CCL4 with a Neutralizing Antibody

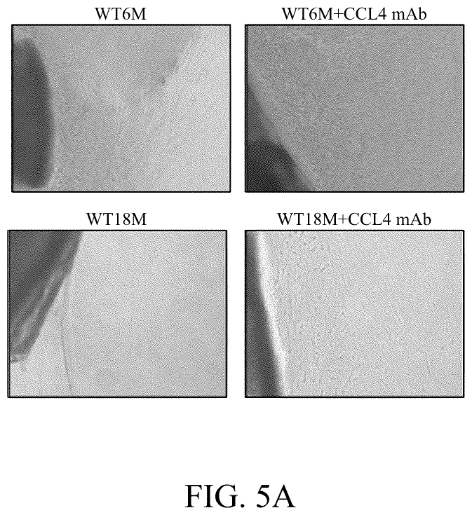

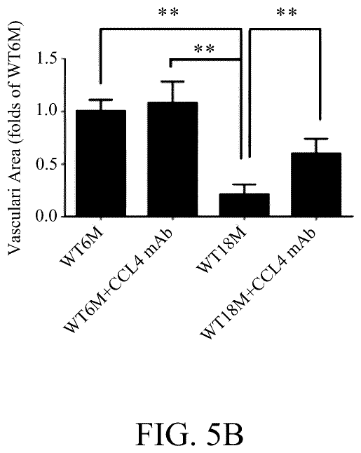

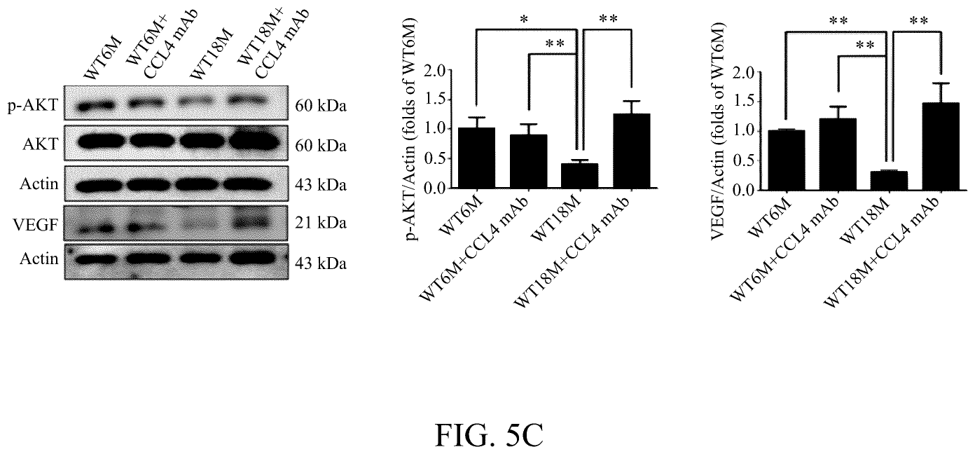

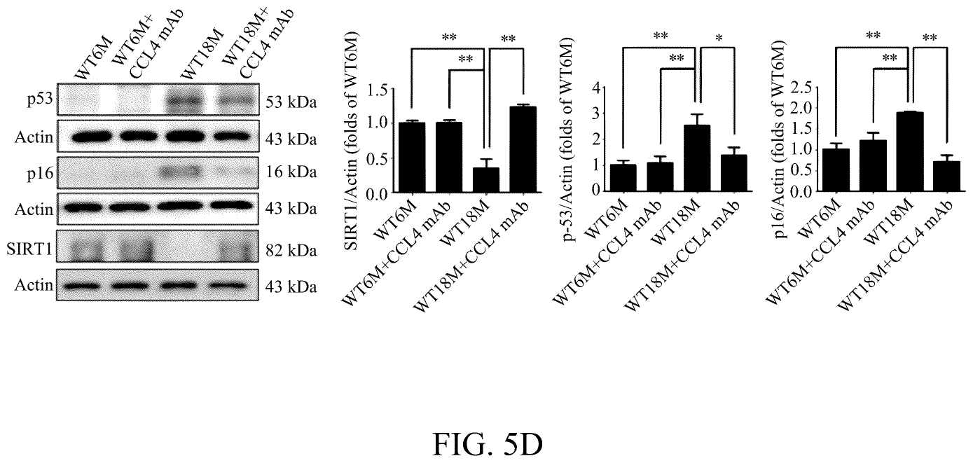

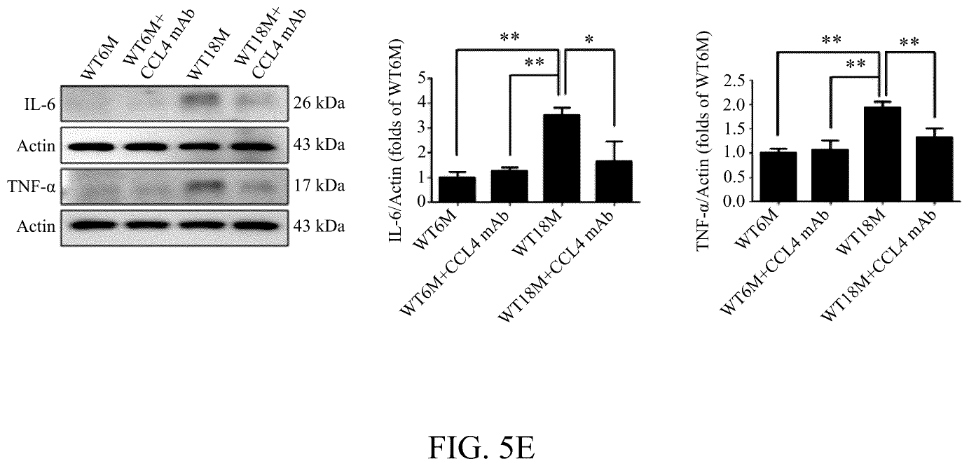

In aged wild-type mice in which CCL4 was inhibited with a neutralizing antibody (WT18M+CCL4 mAb), the area of aortic ring vascular sprouting increased, as compared with aged wild-type mice (WT18M) (FIGS. 5A and 5B). In addition, in the evaluation of the expression of angiogenic, aging, and inflammatory markers in aortic tissues, the aortas of aged wild-type mice in which CCL4 was inhibited with a neutralizing antibody (WT18M+CCL4 mAb) had a higher level of angiogenic factors (p-AKT/VEGF) (FIG. 5C), as well as reversed aging proteins (up-regulated SIRT1/down-regulated p53/down-regulated p16) (FIG. 5D) and a lower level of inflammatory proteins (TNF-α/IL-6) (FIG. 5E), as compared with aged wild-type mice (WT18M).

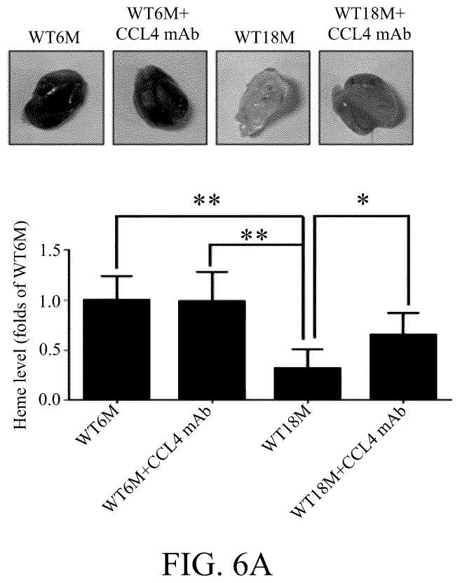

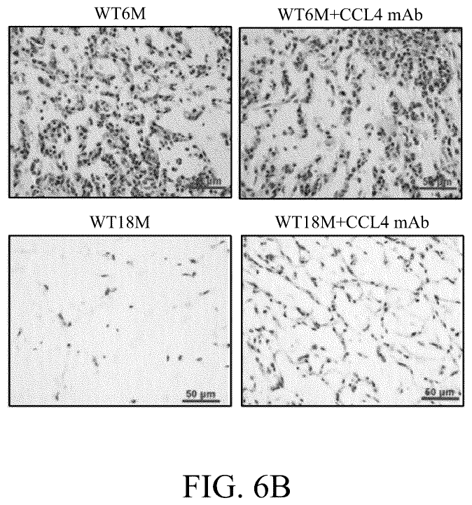

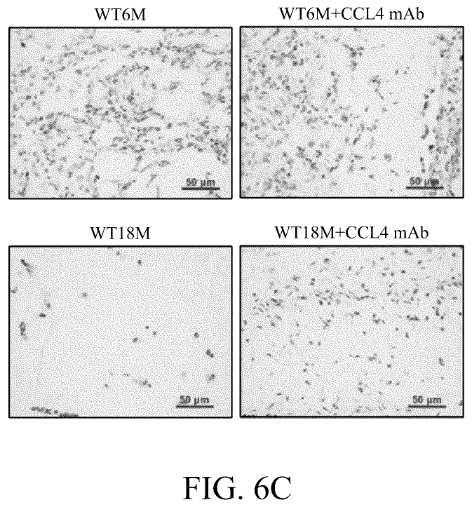

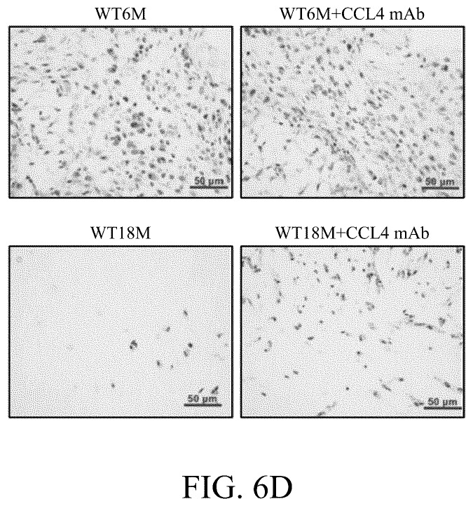

Moreover, the effect of inhibiting CCL4 with a neutralizing antibody on neovascularization was confirmed by Matrigel plug assay in vivo. Lower levels of vascularization and hemoglobin were observed in Matrigel plugs of aged wild-type mice (WT18M), as compared with those in Matrigel plugs of young mice (WT6M). The levels of vascularization and hemoglobin in Matrigel plugs of aged wild-type mice in which CCL4 was inhibited with a neutralizing antibody (WT18M+CCL4 mAb) increased, as compared with aged wild-type mice (WT18M) (FIG. 6A). Increases in the numbers of blood vessels in Matrigel plugs of aged wild-type mice in which CCL4 was inhibited with a neutralizing antibody (WT18M+CCL4 mAb) were also observed in hematoxylin-eosin (H&E) and CD31 staining assay (FIGS. 6B and 6C). In addition, in Matrigel plugs of aged wild-type mice in which CCL4 was inhibited with a neutralizing antibody (WT18M+CCL4 mAb), expression of Ki67 (a marker for cell proliferation) at a level higher than that in aged wild-type mice (WT18M) was observed (FIG. 6D).

It can be seen from the examples described above, the aging factors, the inflammatory factors, and the angiogenic factors in vivo were regulated via inhibiting CCL4, thereby inhibiting vascular aging, including resisting the aging of vascular endothelial cells and/or improving the ability of angiogenesis.

Although the examples of the present disclosure have been illustrated in detail, it is clear to one having ordinary skill in the art that various modifications and variations made to the examples specified without departing from the teachings and features of the present disclosure are still considered to be encompassed within the spirit and scope of the present disclosure. Therefore, such modifications and alternations are encompassed within the spirit and scope of the present disclosure.

What is claimed is:

1. A method for inhibiting vascular aging, including administering a pharmaceutical composition to a subject in need thereof, wherein the pharmaceutical composition includes an effective amount of the CCL4 antagonist and a pharmaceutically acceptable carrier thereof.

2. The method of claim 1, wherein the CCL4 antagonist is at least one selected from the group consisting of an anti-CCL4 antibody or fragment thereof, a CCL4 RNA interference agent, a small molecular CCL4 antagonist, a CCR1 antagonist, a CCR2 antagonist, and a CCR5 antagonist.

3. The method of claim 1, wherein the vascular aging is accompanied with an increase in a CCL4 level.

4. The method of claim 1, wherein the CCL4 antagonist inhibits the vascular aging by reducing a CCL4 level, inhibiting a binding activity of the CCL4 with a receptor thereof, or a combination thereof.

5. The method of claim 4, wherein the inhibition of the vascular aging comprises resisting aging of vascular endothelial cells and/or enhancing an ability of angiogenesis.

6. The method of claim 5, wherein the resisting aging of vascular endothelial cells comprises at least one selected from the group consisting of reducing ROS, reversing aging factors, and reducing inflammatory factors, and the enhancing the ability of angiogenesis comprises up-regulating angiogenic factors.

7. The method of claim 6, wherein the reversing aging factors comprises at least one selected from the group consisting of up-regulating SIRT1, down-regulating p53, and down-regulating p16.

8. The method of claim 6, wherein the inflammatory factors comprise at least one selected from the group consisting of TNF-α, IL-1β, and IL-6.

9. The method of claim 6, wherein the ability of angiogenesis comprises at least one selected from the group consisting of vascularization, an ability of cell migration, and vascular germination.

10. The method of claim 6, wherein the angiogenic factors comprise at least one selected from the group consisting of p-eNOS, p-AKT, VEGF, and SDF-1α.

11. The method of claim 1, wherein the CCL4 antagonist is administrated at an amount of 0.01 mg/kg to 100 mg/kg.

12. The method of claim 1, wherein the pharmaceutical composition is administrated 1 to 25 times per week.


SEQUENCE LISTING (2 sequences)

SEQ ID NO: 1 - DNA, 22 bp, Mus musculus
  source 1..22: mol_type = other DNA
tctccctcctttctcttccgtg

SEQ ID NO: 2 - DNA, 23 bp, Mus musculus
  source 1..23: mol_type = other DNA
tctactcccaatgatggctgacc